(19) United States
(12) Patent Application Publication
(10) Pub. No.: US 2011/0281262 A1
(43) Pub. Date: Nov. 17, 2011

(54) DETECTION OF GERMS ASSOCIATED WITH PERIODONTITIS

(76) Inventors: Peter Johannes Winklehner, Rainbach (AT); Christian Rudolf Mittermayr, Walding (AT)

(21) Appl. No.: 12/734,080

(22) PCT Filed: Oct. 10, 2008
(86) PCT No.: PCT/EP2008/008580

§ 371 Date: May 11, 2010

(30) Foreign Application Priority Data
Oct. 10, 2007 (AT) A 1617/2007

(51) Int. Cl. C12Q 1/68; C12M 1/34

(52) U.S. Cl. 435/6.11; 435/287.2

(57) ABSTRACT

The invention relates to a method for detecting and/or determining bacteria associated with periodontitis from a biological sample with at least one of the oligonucleotides SEQ ID No. 1 to SEQ ID No. 5 as well as a microfluidic device for detecting and/or determining at least one germ associated with periodontitis from a biological sample comprising a carrier consisting of at least one base part with a surface and at least one oligonucleotide bound to the carrier surface or nucleic acid molecule, in which the oligonucleotide represents at least one sequence of SEQ ID No. 1 to SEQ ID No. 5.

The present invention describes a method, a microfluidic device and a kit for detecting and/or determining bacteria associated with periodontitis in a biological sample.

Periodontitis is a bacterial inflammation which manifests itself as mostly irreversible damage to the supporting tissue of the tooth (periodontium).

Periodontitis is triggered by bacterial plaque (film on the teeth), a glutinous adhesive biofilm. Its main distinguishing feature is the loss of bone which can be seen on an X-ray in the case of periodontitis, in which deepened gum pockets with gingivitis are caused by the inflammatory swelling of the gingiva. In both gingivitis and periodontitis bacterial metabolic and decay products are released from the biofilm, which trigger the defence reactions of the body. The body's immune system contributes significantly to the tissue damage as it attempts to remove the bacteria. The immunity response consists of a diverse sequence of reactions and actions involving various different inflammatory substances and cells. Enzymes are also formed which are intended to destroy the bacteria but also cause tissue damage. The end result is a loss of connective tissue and bone. The reaction to the bacteria results in bleeding gums, the formation of pockets, receding gums and in the end the loosening and loss of teeth.

Over the age of thirty five more people lose teeth through periodontitis than through caries. It is largely unrecognised that diseased gums can also severely affect other organs. Periodontitis is a very common disease in which the gums or the supporting tissue of the teeth are damaged over the years due to inadequate oral hygiene. If the periodontitis is not treated there is a chance that teeth may be lost.

In most cases it develops as a chronic disease. It mainly affects adults, is rarely painful and causes the teeth to loosen only after many years of progression without the affected person being aware of the problem. To a certain degree the margin of the gum protects bacteria from the self-cleaning oral cavity with the tongue and saliva. In healthy people the so-called marginal epithelium with its adhesion to the enamel guarantees a continual surface between the gum and the tooth. If the plaque is not removed carefully from these niches, the excretion products of the microorganisms (exotoxins) attack the marginal epithelium and some bacteria are capable of passing through the epithelium. The body reacts to such attacks by importing antibodies from the blood. Gradually the intruders are destroyed and phagocyted. At the same time various endotoxins are released. Both the exotoxins and endotoxins and several decay products from antibodies act as irritants. In order to protect the surrounding tissue from these irritants and prevent the inflammation from advancing into the jaw the body also activates osteoclasts. Their purpose is the targeted decomposition and alteration of bone tissue.

If the body has good immunity the microorganisms can be prevented from penetrating into the jaw for a long time. However, the power relationships in this struggle are very variable. A worsening of the body's defence system, a significant increase in bacteria, or a change in the aggression of the microorganisms can result in further progression of the inflammatory process. Over time this can lead to a continual loss of bone, which can only be stopped by the complete removal of the stimuli. On X-ray images the bone loss appears to be mainly horizontal, as in the rest phases of the inflammation the osteoclasts shape the fissured bone tissue and adapt to the new conditions. Owing to the slow and gradual progression of the disease this form of inflammation is defined as chronic periodontitis. This differs from aggressive periodontitis which leads rapidly to comprehensive bone loss and sometimes even occurs in children.

Of the 500 different species of bacteria that can live in the oral cavity only a small number are periodontal pathogens. The latter are defined as principle germs and form so-called clusters which exhibit specific socialisation behaviour. They are obligate or facultative anaerobic, gram-negative, black pigmented bacteria types, such as the so-called red complex group (porphyromonas gingivalis, treponema denticola and tannerella forsythenisis as well as actinobacillus actinomycetemcomitans subtype B).

Currently the treatment consists of removing the inflammation from the gum and the supporting tissue of the tooth and removing the plaque and tartar as well as any inflammatory factors. The treatment consists of different phases, in which different procedures are carried out. The first phase consists of a comprehensive diagnosis to determine the type, severity and progression of the disease. In addition to X-rays a clinical assessment of the overall state of the teeth is carried out. In the meantime in many cases supplementary microbiological tests are performed (detection of specific periodontal pathogenic bacteria). If the periodontitis is so pronounced that antibiotics have to be prescribed it is advantageous to establish the type of bacteria to ensure a more specific treatment.

If periodontitis is not treated it almost always results in tooth loss and thus also affects the aesthetic appearance and functioning of the teeth. Furthermore, periodontitis is a risk factor for general medical diseases. There is a scientifically proven link between periodontal diseases and an increased risk of heart attack and diseases of the rheumatic system. More recent investigations have also shown that untreated periodontitis increases the risk of premature birth sevenfold and periodontitis can also be a cause of low birth weight.

From AT 411 174 B a method and a kit for analysing nucleic acids are known, which are used for identifying bacteria associated with periodontitis. In this case, an oligonucleotide is applied, with a sequence that is complementary to the target nucleic acid sequence, onto the surface of a carrier, which has at least one predefined analysis area and predefined control area. Oligonucleotides are used for identifying various bacteria associated with periodontitis, which preferably consist of 10 to 120 nucleotides. A biochip is used as the carrier on which the specific oligonucleotides are spotted. Before the biological sample comes into contact with the biochip, by means of specific primer sequences the bacteria DNA in the biological sample is amplified.

DE 699 24 741 T2 describes a composition for producing a medicine for preventing and treating periodontitis. In this case periodontitis is controlled effectively by using a varnish containing an antimicrobial agent. An antimicrobial composition is used which comprises a physiologically compatible clear varnish base and an antimicrobial agent dissolved therein.

Also from DE 101 54 290 A1 a method is known for detecting bacteria associated with periodontitis and caries. In particular, the invention relates to hybridisation methods and amplification methods as well as combined amplification/hybridisation methods with sequence-specific probes or primers. For the amplification preferably a polymerase chain reaction is used. In the simplest method of detecting the nucleic acid to be detected the amplificate is cut specifically e.g. by digestion with a restriction enzyme and the resulting fragments are analysed on an agarose gel. Also hybridisation systems are widespread, in which hybridisation occurs so that either the composition which contains the amplification product or a portion thereof or the probe is immobilised in a fixed phase and is brought into contact with the other respective hybridisation partner. In the fixed phase different materials can be used, such as for example a microtitre plate. The target sequence can also be hybridised previously in solution with a catching probe and then the catching probe is bound to a fixed phase. As a rule at least one probe or at least one primer is marked in the case of amplification or nucleic acid to be detected.

Therefore, the objective of the present invention is to provide a method and a device for detecting and/or determining bacteria associated with periodontitis, in which the result is provided rapidly and the device is simple to produce and manipulate.

The objective according to the invention is achieved independently by a method for detecting and/or determining bacteria associated with periodontitis in a biological sample with at least one of the oligonucleotides SEQ ID No. 1 to SEQ ID No. 5, a microfluidic device comprising a carrier with at least one base part and/or cover part with a surface and at least one oligonucleotide or nucleic acid molecule bound to the carrier surface, i) oligonucleotide SEQ ID No. 1 to SEQ ID No. 8, ii) oligonucleotide which has a nucleic acid sequence that is mutated in relation to a oligonucleotide SEQ ID No. 1 to SEQ ID No. 8, such as by the addition or deletion of 1 to 10 nucleotides or a substitution of 1 to 3 nucleotides in one of the nucleotide sequences shown in SEQ ID No. 1 to SEQ ID No. 8, iii) oligonucleotide, which has a nucleotide sequence, which is complementary at least in areas to the nucleotide sequence SEQ ID No. 1 to SEQ ID No. 8, and a kit comprising at least one carrier, such as a microfluidic device, with at least one base and/or cover part with a surface with at least one immobilised oligonucleotide and nucleic acid sequence containing the at least i) oligonucleotide SEQ ID No. 1 to SEQ ID No. 8 ii) oligonucleotide which has a nucleic acid sequence that is mutated in relation to a oligonucleotide SEQ ID No. 1 to SEQ ID No. 8, such as by the addition or deletion of 1 to 10 nucleotides or a substitution of 1 to 3 nucleotides in one of the nucleotide sequences shown in SEQ ID No. 1 to SEQ ID No. 8, iii) oligonucleotide, which has a nucleotide sequence, which is complementary at least in areas to the nucleotide sequence SEQ ID No. 1 to SEQ ID No. 8, and at least one container with a universal solution. It has proved to be advantageous in this case that by means of the species-specific sequences the exact identification of the bacteria is possible and thus a clear allocation can be made to a pathological model.

In one development the method comprises the following steps: a) transfer of the biological sample into a universal solution in a container, which contains at least one sandwich oligonucleotide, preferably oligonucleotide SEQ ID No. 6, b) denaturising the sample, in particular by heating the contents of the container, c) transfer of at least one portion of the solution into or onto a carrier with at least one immobilised oligonucleotide SEQ ID No. 1 to SEQ ID No. 5 and possibly SEQ ID No. 7 and/or 8, d) hybridisation and possibly incubation, and e) detection. It has proved to be advantageous if the biological sample is marked by the oligonucleotide SEQ ID No. 6, which binds to a highly-conserved region, and by means of the species-specific probes, which are immobilised on the carrier, can be bound and detected and assigned to specific bacteria. By means of control sequences, such as oligonucleotide SEQ ID No. 7 and SEQ ID No. 8, the hybridisation and generally the method can be controlled.

By applying at least one additional reaction solution, such as a marker solution, enzyme solution, washing solution, colour reaction solution, into or onto the carrier the method is improved, in that for example during the detection process disruptive signals can be avoided by the washing solution, or by means of a corresponding enzyme and colour reaction solution the detection limit can be lowered.

The analysis of a nucleic acid contained in the biological sample is performed by hybridisation with at least one probe, where the probe is selected from a group comprising i) oligonucleotide SEQ ID No. 1 to SEQ ID No. 8, ii) oligonucleotide which has a nucleic acid sequence that is mutated in relation to a oligonucleotide SEQ ID No. 1 to SEQ ID No. 8, such as by the addition or deletion of 1 to 10 nucleotides or a substitution of 1 to 3 nucleotides in one of the nucleotide sequences shown in SEQ ID No. 1 to SEQ ID No. 8, iii) oligonucleotide, which has a nucleotide sequence, which is complementary at least in areas to the nucleotide sequence SEQ ID No. 1 to SEQ ID No. 8. It is advantageous in this case that the detection and/or determining of bacteria associated with periodontitis can be achieved very rapidly, preferably in less than 15 min. It is also an advantage that the detection method is very sensitive and bacteria can be detected in a number of less than 10⁵ bacteria. As well as the rapid completion time of this method the latter is also very economical and easily to handle in that no special laboratory equipment is required. The method according to the invention can thus also be performed in a dentist's surgery and the result can be given to the patient at the same appointment. Confirmation of the analyte is possible by visual evaluation.

A capillary system can be used as the carrier, such as a test strip or microfluidic device, which means that a simple device can be used for detecting germs associated with periodontitis, as no additional means, such as for example a mains-operated device, are necessary for adjusting and/or controlling the current and/or voltage for transporting the sample.

An oligonucleotide or nucleic acid molecule can be used as the probe which is a DNA, RNA, PNA, LNA-molecule or a mixed form, as LNA-oligonucleotides and PNA-molecules correspond to the Watson-Crick-model and hybridise on complementary oligonucleotides. LNA/DNA- or LNA/RNA-duplex molecules exhibit increased thermal stability compared to similar duplex molecules, which are formed exclusively by DNA or RNA.

It has proved to be an advantage if the biological sample is a subgingival plaque sample, in particular from the periodontium, as this can be taken by means of non-invasive methods and can be provided by any one at any time.

Furthermore, it is an advantage if the surface of the microfluidic device facing the biological sample is hydrophilised at least in some areas, in particular modified by plasma polymerisation, which allows the capillary fluid transport of the biological sample on a microfluidic device or at least facilitates the latter. In this case, it is particularly advantageous for the capillary fluid transport to be continuous and for there to be no separation of the fluid flow in the canal of the microfluidic device.

At least one sterile sample taking element, preferably a paper tip, curette, spatula or the like is moved to the bottom of a pocket and if necessary left there for a while, whereby a biological sample can be taken by means a simple but not a sharp or pointed object, which does not cause any injury and does not damage the possibly much weakened or affected tissue.

The universal solution preferably contains a chaotropic reagent, such as guanidinium salts, e.g. guanidinium isothiocyanate, formamide, urea, perchlorate, thiocyanate, trichloroacetate, nitrate, iodide, etc. in a concentration of 0.1 M to 10 M, whereby stringent conditions for hybridisation at room temperature are created and furthermore cell lysis can occur. By using guanidinium thiocyanate or urea the use of teratogenic substances becomes obsolete and harm to the person handling the solution is prevented.

It has also proved to be an advantage if the universal solution contains a marker, in particular the marked oligonucleotide SEQ ID No. 6 for the biological sample, whereby in one operating step both the lysis of the biological sample and the marking of the nucleic acid can be performed.

Many of the steps, in particular the hybridisation, can be performed at room temperature, which supports the option of it being performed as a chairside test and a heat source is only required for performing one step of the operation (lysis).

According to the invention the container is heated to a temperature of at least 50° C., whereby on the one hand the cell decomposition of the biological sample is supported and on the other hand nucleic acid molecules are denatured.

Preferably, the still heated biological sample is transferred onto the carrier, preferably the microfluidic device, by means of a pipette or capillary, which ensures that the biological sample remains in the denatured state.

There can also be an incubation period of 1 min to 10 min, which is a sufficient amount of time to find complementary nucleic acid molecules.

The enzyme, washing and colour reaction solution are applied respectively by a dropper and the microfluidic device with the respective solution is preferably incubated for a period of 5 sec to 10 min, during which the reactions necessary for the visual detection of the analyte can take place.

In a preferred exemplary embodiment the detection is performed by colorimetry, whereby tetramethylbenzidine is added, preferably dissolved in polyvinylpyrrolidone, to commercially available tetramethylbenzidine reaction mixtures for the colour reaction, whereby by the formation of complementary colours the visual signal is intensified

It is also the case that the method can be used for the diagnosis and/or early identification of disease, the preliminary stages of disease, risks of disease and/or diseased alterations which are associated with the bacteria causing periodontitis, so that the risk of developing periodontitis can be recognised early.

By selecting the specific sequences for the bacterial strains tannerella forsythensis, porphyromonas gingivalis, prevotella intermedia, treponema denticola and hemophilus actinomycetemcomitans it is ensured that bacteria actually associated with periodontitis are detected in the oral cavity or tooth pocket.

By using the universal probe SEQ ID No. 8 as far as possible all of the bacteria in the oral cavity are detected. This ensures that all of the bacteria located in the tooth pocket can be detected that have no complementary sequence to the oligonucleotides SEQ ID No. 1 to SEQ ID No. 7.

It is also possible to provide a control, such as a positive, negative, orientation, hybridisation and/or colour reaction control or the like, on the carrier surface of the microfluidic device, whereby it can be proved, that the test has actually worked, but as for example with bacteria-specific probes there was no binding and no periodontitis-associated bacteria occur in the biological sample.

It is also advantageous for the kit to contain at least one additional reaction solution, such as a washing, enzyme, colour reaction solution and/or the like, whereby all of the reagents necessary for performing the analysis and/or determining and detecting bacteria associated with periodontitis are included in the kit and no additional reagents have to be bought separately.

For the user it is only necessary to provide a heat source for the biological sample, to reach a temperature of at least 50° C. for the cell breakdown or the denaturisation of the nucleic acids. The method according to the invention enables the detection of bacteria associated with periodontitis without first having to perform amplification, as known from the prior art.

First of all, it should be noted that in the variously described exemplary embodiments the same parts have been given the same reference numerals and the same component names, whereby the disclosures contained throughout the entire description can be applied to the same parts with the same reference numerals and same component names. Also details relating to position used in the description, such as e.g. top, bottom, side etc. relate to the currently described and represented figure and in case of a change in position should be adjusted to the new position. Furthermore, also individual features or combinations of features from the various exemplary embodiments shown and described can represent in themselves independent or inventive solutions.

All of the details relating to value ranges in the present description are defined such that the latter include any and all part ranges, e.g. a range of 1 to 10 means that all part ranges, starting from the lower limit of 1 to the upper limit 10 are included, i.e. the whole part range beginning with a lower limit of 1 or above and ending at an upper limit of 10 or less, e.g. 2 to 9, or 4 to 7 or 5 to 6.

A subgingival plaque sample is taken from the deepest point of the periodontal pocket, possibly with bleeding on exploration and/or suppuration, preferably in each quadrant. Acutely bleeding and/or heavily suppurating points should be avoided. The biological sample is preferably taken by the dentist or assistant with a paper tip from the bottom of the pocket. Other means can also be used for taking the sample such as for example a cotton bud, swab, curette etc.

Also several biological samples can be taken and added jointly to the method according to the invention. It has proved to be advantageous to use a maximum of 2 paper tips per test sequence, whereby a maximum of 2 different sample sites are combined into a mixed sample and applied onto the carrier. If more points are to be tested preferably the additional paper tips are fed into further sample tubes and a new carrier is used. If the method according to the invention is to be used for screening purposes the sample is preferably taken by curette.

After draining and removing the supragingival plaques the removal site is isolated by cotton wool rolls in order to remove subgingival plaque. Then the sterile paper tips are moved by a pair of tweezers to the bottom of the pocket and left there for about 15 seconds (sec). The paper tips are again removed by the tweezers and placed into the sample tube. In the sample tube the universal solution is already provided or the latter is added. The paper tip brought into contact with the biological sample has to be immersed in the solution in the sample tube.

Preferably a 500 μl sample tube is used, which contains an aliquot of the universal solution, preferably between 10 μl and 300 μl. The sample tube is sealed and mixed, e.g. shaken forcefully and/or vortexed, stirred, resuspended, etc. Lastly, the liquid at the bottom of the sample tube is collected and the liquid together with the biological sample is heated in the sample tube. The sample tube is preferably heated to a temperature selected from a range with a lower limit of 72° C. and an upper limit of 100° C., preferably 90° C., in particular 95° C. In order to achieve the complete lysis and denaturisation of the biological sample it is preferably heated for a period of at least 10 sec to max. 5 min.

After heating the hot liquid is removed and applied onto the carrier, preferably the sample feeding opening of the microfluidic device, which is described in the Austrian patent application A01618/2007 of the applicant and the content of which is included within the scope of this application.

The microfluidic device comprises at least one base part and/or cover part. A canal is arranged in the base part and/or cover part. The canal connects the sample feeding opening with a widening at the opposite end of the sample feeding opening.

The removal of the sample is preferably performed by means of a pipette or capillary. The biological sample or liquid can be suctioned by capillary force into the canal of the microfluidic device. There is then an incubation period of at least 10 sec, preferably at least 1 min.

After this incubation period an enzyme solution is applied into the sample feeding opening of the microfluidic device. Then after an incubation period of at least 10 sec, whereby in this period the enzyme solution is also suctioned into the canal, a washing solution is added into the sample feed opening of the microfluidic device and incubated again, to allow the suctioning of the liquid into the canal.

Lastly, the colour reaction solution is added to the sample feeding opening of the microfluidic device and the colour reaction can be continued until there is an optimum display of the signal.

The application of the enzyme solution, the washing solution and the colour reaction solution can be performed by means of a pipette or dropper. Depending on the duration of the individual incubation steps the method according to the invention is completed after 1 min to 2 min. However, the incubation times can also be extended so that the result of the analysis appears only after 10 or more minutes. The result of the analysis can be read visually or scanned into a desktop scanner and evaluated.

The universal solution includes at least one chaotropic reagent and at least one oligonucleotide. The universal solution can include for example guanidiniumthiocyanate, tris-HCl, DNA and a marked oligonucleotide. Similar solutions are known for example from U.S. Pat. No. 5,334,501.

The enzyme solution includes a peroxidase conjugate stabiliser and streptavidin peroxidase.

In order to produce the washing solution sodium chloride, sodium citrate and proclin 200 is weighed and provided with water.

To produce the colour reaction solution the TMB and TMB/PVP-solution and proclin 200 are mixed together and also a commercially obtainable deposit forming TMB-solution can be used.

For the oligonucleotide sequences preferably the following sequences are used, whereby at least 15 of the given consecutive nucleotides of the respective oligonucleotide, their complementary and/or mutated sequences or the like are used.

The sandwich-probe is used for labelling the 16 S rRNA of the biological sample. It is labelled with biotin at 5′ end. The sandwich-probe has the sequence 5′-CATCG AATTA AACCA CATGY TCCWC CGCTT GT-3′ (SEQ ID No. 6). The sandwich probe binds with the biological sample and because the sandwich probe itself is marked, the biological sample is also marked. Preferably, the biological sample is marked by means of the oligonucleotide SEQ ID No. 6 in the universal solution. The oligonucleotide SEQ ID No. 6 itself bears the marking/label. Said marking can either be detected itself (directly) or detected by additional binding steps (indirectly).

As the control probe sequence for the hybridisation control an oligonucleotide is used with the sequence 5′-ACAAG CGGWG GARCA TGTGG TTTAA TTCGA TG-3′ (SEQ ID No. 7). SEQ ID No. 7 is partly complementary to SEQ ID No. 6 and therefore is used as a control and is preferably immobilised on the carrier.

As a control for the enzyme conjugate a biotin linked oligonucleotide is used with any sequence or a biotin derivative reacting with the surface.

The oligonucleotides SEQ ID No. 1 to 5 are bacteria-specific probes. For the detection of the bacterial strain tannerella forsythensis an oligonucleotide is used with the sequence 5′-AAGAA AGCTC TCACT CTCCG TCGTC TA-3′ (SEQ ID No. 1).

As a specific probe for porphyromona gingivalis an oligonucleotide is used with the sequence 5′-CGCTG TGGAA GCTTG ACGGT ATATC GCAAA CTC-3′ (SEQ ID No. 2).

For the detection of the bacterial strain prevotella intermedia an oligonucleotide is used with the sequence 5′-AGTCA ACATC TCTGT ATCCT GCGTC TGCA-3′ (SEQ ID No. 3).

As a specific oligonucleotide for the bacterial strain treponema denticola an oligonucleotide is used with the sequence 5′-AAGA GCCGT ATTGC TACGC TGCCA TATCT CTA-3′ (SEQ ID No. 4).

As a bacterial strain specific probe for the detection of hemophilus actinomycetemcomitans an oligonucleotide is used with the sequence 5′-GTCTC AAGC TCCCT AAGGC TCAAA CCCAT C-3′ (SEQ ID No. 5).

The universal probe, which is to detect as far as possible all of the bacterial strains found in the mouth cavity and also functions as a control probe, has the sequence 5′-CCCGT CAATT CMTTT GAGTT TYAMC STTGC-3′ (SEQ ID No. 8). The universal probe is preferably also immobilised on the carrier.

To mark the biological sample a labelling reagent is used, which preferably consists of a label, a reactive group and a link between the two groups. The reactive groups are psoralens, aryl azides, reactive groups from the mustard compounds (label-IT), cisplatin complexes (Universal Label System), etc. or for example a sandwich-oligonucleotide is used which hybridises in a position different than the specific sample position to the target.

In addition to the already described visual, in particular colorimetric detection methods with tetramethylbenzidine (TMB), also other reagents such as diaminobenzidine (DAB), diaminobenzidines with a subsequent silver mirror reaction, silver enhancement (amplification) on gold nanoparticles, standard substrates for horseradish-peroxidase and alkali phosphatase, etc. can be used. A detection method with a luminescent basis is achieved for example for light generation by the breakdown of luminol by horseradish-peroxidase. The detection of this light is performed using photographic paper or photographic diodes.

In order to improve colorimetric detection methods, in particular the tetramethylbenzidine methods, the following is proposed: the oxidation of TMB by HRP and hydrogen peroxide runs in two stages. Firstly, a complex is formed from an oxidised and a non-oxidised TMB-molecule, which is coloured intensively blue. In a continuing reaction TMB is oxidised into a yellow product. In commercially obtainable TMB-reaction mixtures hydrogen peroxide is available in molar excess. The reaction therefore runs beginning with a blue intermediate product to a yellow end product. The colour thus changes from blue via green to yellow. By adding TMB to this reaction mixture the reaction can be stopped at the blue intermediate product. In addition TMB dissolved in 1% polyvinylpyrrolidone is added. The reaction stops at the blue intermediate product, which is more clearly visible to the eye

As already mentioned, the surface of the substrate has to be hydrophilic, so that the liquid can be suctioned into the canal and at the same time the nucleic acid binding is improved. It has also proved to be advantageous if the hydrophilisation method employed can be used for the modification of plastic surfaces. This necessary hydrophilisation is achieved both by plasma polymerisation and also for example by surface modifications known from the prior art, such as e.g. from the company PolyAn. With an unfunctionalised surface the liquid remains in the sample feeding opening and there is no capillary liquid transport.

In addition, by means of the hydrophilisation of the surface of the carrier of the microfluidic device, in particular plasma polymerisation, a yellow colouring of the otherwise white surface of the carrier of the microfluidic device is produced. Said yellow colouring amplifies the signal as the colour of the precipitate, as already mentioned, is blue. With the complementary colour yellow a darker colouring of the precipitate is obtained.

In comparison trials between plasma polymerisation and the treatment of the carrier surface with Polyan for hydrophilisation it was established that plasma polymerisation achieved at least equally good immobilisation properties and hybridisation properties of the immobilised DNA-samples. In addition, it was established that by means of plasma polymerisation a lower unspecific adsorption of nucleic acids as well as proteins occurred on the surface of the microfluidic device. The surface modification by means of plasma polymerisation is described in the Austrian patent application A01619/2007 of the applicant and the content of the latter is thus included within the scope of this application.

### EXAMPLE OF APPLICATION

The sample taken by a dentist with a paper tip is put into a 500 μl sample tube and 60 μl universal solution is added. The sample tube is sealed and shaken forcefully for 20 s. By means of a shaking movement the liquid is collected in the tip of the sample tube and heated for 1 min in a heating block to 95° C. By means of Pasteur pipette the 95° C. liquid is removed and 1 drop is added to the microfluidic device. After an incubation period of 1 min 20 μl enzyme solution is dropped into the feed opening of the platform. Once this has been suctioned (ca. 30 s), 20 μl washing solution is added and there is a waiting period until the liquid has been completely suctioned. Lastly, the colour reaction solution is added and the colour reaction is waited for until there is optimum visibility of the bands. The reaction is completed after about 2 min and the result of the analysis is read visually and scanned by a desktop scanner.

The exemplary embodiment shows a possible embodiment variant of the method according to the invention, whereby it should be noted at this point that the invention is not restricted to the embodiment variants shown in particular, but rather various different combinations of the individual embodiment variants are also possible and this variability, due to the teaching on technical procedure, lies within the ability of a person skilled in the art in this technical field. Thus all conceivable embodiment variants, which are made possible by combining individual details of the embodiment variants shown and described, are also covered by the scope of protection.

The problems addressed by the independent solutions according to the invention can be taken from the description.

1-17. (canceled)

18. Method for detecting and/or determining bacteria associated with periodontitis from a biological sample with at least one of the oligonucleotides SEQ ID No. 4 to SEQ ID No. 5 and possibly SEQ ID No. 1 to SEQ ID No. 3, comprising the steps: i) recovery of the biological sample, ii) denaturization of the biological sample, in particular cells, iii) hybridization of the lysated sample with at least one of the oligonucleotides SEQ ID No. 4 to SEQ ID No. 5 and possibly SEQ ID No. 1 to SEQ ID No. 3 on a carrier and iv) detection of the result.

19. Method according to claim 18, wherein the detection and/or determining comprises the steps: a) transferring the biological sample into a universal solution containing at least one chaotropic reagent in a container, which contains at least oligonucleotide SEQ ID No. 6, b) denaturizing the sample, in particular by heating the container, c) transferring at least one portion of the solution into or onto a carrier with at least one immobilized oligonucleotide of SEQ ID No. 4 to SEQ ID No. 5 and possibly SEQ ID No. 1 to SEQ ID No. 3, SEQ ID No. 7 and/or 8, d) hybridization and possibly incubation and e) detection.

20. Method for detecting and/or determining bacteria associated with periodontitis from a biological sample, wherein the analysis of a nucleic acid contained in the biological sample is performed by hybridization with at least one probe, wherein the probe is selected from a group containing: i) oligonucleotide SEQ ID No. 4 to SEQ ID No. 5 and possibly SEQ ID No. 1 to 3 or SEQ ID No. 6 to 8, ii) oligonucleotide which has a nucleotide sequence that is mutated in relation one of the oligonucleotides SEQ ID No. 4 to SEQ ID No. 5 and possibly SEQ ID No. 1 to SEQ ID No. 3 or SEQ ID No. 6 to 8, in particular by the addition or deletion of 1 to 10 nucleotides or a substitution of 1 to 3 nucleotides in one of the nucleotide sequences represented in SEQ ID No. 1 to SEQ ID No. 8 or iii) oligonucleotide, which has a nucleotide sequence, which is complementary at least in areas to the nucleotide sequence SEQ ID No. 4 to SEQ ID No. 5, and possibly SEQ ID No. 1 to SEQ ID No. 3 or SEQ ID No. 5 to 8.

21. Method according to claim 18, wherein as the carrier a capillary system is used, preferably a test strip or microfluidic device.

22. Method according to claim 18, wherein the oligonucleotide used as a probe or nucleic acid molecule is a DNA, RNA, PNA, LNA molecule or a mixed form thereof.

23. Method according to claim 18, wherein the universal solution contains chaotropic reagents, in particular guanidinium salts such as guanidinium-isothiocyanate, formamide, urea, perchlorate, thiocyanate, trichloroacetate, nitrate, iodide, preferably in a concentration of 0.1 M to 10 M.

24. Method according to claim 18, wherein in the universal solution the marking of the biological sample is performed, in particular by the marked oligonucleotide SEQ ID No. 6.

25. Method according to claim 18, wherein the hybridization is performed at room temperature.

26. Method according to claim 18, wherein the denaturization of the sample is performed at a temperature selected from a range with a lower limit of 50° C. and an upper limit of 100° C.

27. Method according to claim 26, wherein the transfer of the preferably still heated biological sample on the microfluidic device is performed by means of a pipette or capillary.

28. Method according to claim 18, wherein the biological sample is applied without previous amplification on the microfluidic device.

29. Microfluidic device for detecting and/or determining at least one germ associated with periodontitis from a biological sample comprising a carrier consisting of at least one base part with a surface and at least one oligonucleotide or nucleic acid molecule bound onto the carrier surface, wherein i) the oligonucleotide is at least one sequence of SEQ ID No. 4 to SEQ ID No. 5 and possibly SEQ ID No. 1 to SEQ ID No. 3 or SEQ ID No. 6 to 8, ii) the oligonucleotide has a nucleotide sequence that is mutated in relation to one of the oligonucleotides SEQ ID No. 4 to SEQ ID No. 5 and possibly SEQ ID No. 1 to SEQ ID No. 3 or SEQ ID No. 6 to 8, in particular the addition or deletion of 1 to 10 nucleotides or a substitution of 1 to 3 nucleotides in one of the nucleotide sequences represented in SEQ ID No. 1 to SEQ ID No. 8, or iii) the oligonucleotide has a nucleotide sequence, which is complementary at least in areas to the nucleotide sequence SEQ ID No. 4 to SEQ ID No. 5 and possibly SEQ ID No. 1 to SEQ ID No. 3 or SEQ ID No. 6 to 8.

30. Microfluidic device according to claim 29, wherein at least one control nucleotide, preferably positive, negative, orientation, hybridisation, color reaction control oligonucleotide is arranged on the carrier surface.

31. Kit for detecting and/or determining at least one germ associated with periodontitis from a biological sample comprising at least one carrier, preferably a microfluidic device, with i) at least one immobilized oligonucleotide or nucleic acid sequence containing the at least one sequence of SEQ ID No. 4 to SEQ ID No. 5 and possibly SEQ ID No. 1 to SEQ ID No. 3 or SEQ ID No. 6 to 8, ii) oligonucleotide, which has a nucleotide sequence which is mutated relative to one of the oligonucleotides SEQ ID No. 4 to SEQ ID No. 5 and possibly SEQ ID No. 1 to SEQ ID No. 3 or SEQ ID No. 6 to 8, in particular by the addition or deletion of 1 to 10 nucleotides or a substitution of 1 to 3 nucleotides in one of the nucleotide sequences represented in SEQ ID No. 1 to SEQ ID No. 8 or iii) oligonucleotide, which has a nucleotide sequence, which is complementary at least in parts to the nucleotide sequence SEQ ID No. 4 to SEQ ID No. 5 and possibly SEQ ID No. 1 to SEQ ID No. 3 or SEQ ID No. 6 to 8 and at least one container with a universal solution containing at least one chaotropic reagent.


SEQUENCE LISTING (8 sequences)

SEQ ID NO: 1 - DNA, 27 bp, artificial Sequence
  Description of the artificial sequence oligonucleotide
aagaaagctctcactctccgtcgtcta

SEQ ID NO: 2 - DNA, 33 bp, artificial sequence
  Description of the artificial sequence oligonucleotide
cgctgtggaagcttgacggtatatcgcaaactc

SEQ ID NO: 3 - DNA, 29 bp, artificial sequence
  Description of the artificial sequence oligonucleotide
agtcaacatctctgtatcctgcgtctgca

SEQ ID NO: 4 - DNA, 32 bp, artificial sequence
  Description of the artificial sequence oligonucleotide
aagagccgtattgctacgctgccatatctcta

SEQ ID NO: 5 - DNA, 31 bp, artificial sequence
  Description of the artificial sequence oligonucleotide
gtctcaaagctccctaaggctcaaacccatc

SEQ ID NO: 6 - DNA, 32 bp, artificial sequence
  Description of the artificial sequence oligonucleotide
catcgaattaaaccacatgytccwccgcttgt

SEQ ID NO: 7 - DNA, 32 bp, artificial sequence
  Description of the artificial sequence oligonucleotide
acaagcggwggarcatgtggtttaattcgatg

SEQ ID NO: 8 - DNA, 30 bp, artificial sequence
  Description of the artificial sequence oligonucleotide
cccgtcaattcmtttgagtttyamcsttgc